(19) United States
(12) Patent Application Publication
(10) Pub. No.: US 2019/0365322 A1
(43) Pub. Date: Dec. 5, 2019

(54) GARMENT WITH BIOLOGICAL SENSOR ATTACHED THERETO

(71) Applicant: TOYOTA JIDOSHA KABUSHIKI KAISHA, Toyota-shi (JP)

(72) Inventors: Kenichi HIROMOTO, Okazaki-shi (JP); Hitoshi YAMADA, Nagakute-shi (JP); Shintaro YOSHIZAWA, Nagoya-shi (JP)

(73) Assignee: TOYOTA JIDOSHA KABUSHIKI KAISHA, Toyota-shi (JP)

(21) Appl. No.: 16/389,294

(22) Filed: Apr. 19, 2019

(30) Foreign Application Priority Data
May 31, 2018 (JP) 2018-105020

(51) Int. Cl. A61B 5/00

(52) U.S. Cl. CPC A61B 5/6804; A61B 5/6823; A61B 2560/0468; A61B 2562/222; A61B 2562/04

(57) ABSTRACT

The present disclosure provides a garment including a plurality of biometric sensors and adapted to cover a body part, a shoulder, and an upper limb of a subject, the plurality of biometric sensors being attached to the garment so that when the subject wears the garment each of them comes into contact with a respective one of a plurality of testing places, in which biometric sensors that are adjacent to each other across a shoulder joint are connected to each other by a wiring cable that passes above an acromial end, and biometric sensors that are adjacent to each other across an elbow joint are connected to each other by a wiring cable that passes an outer side of a lateral epicondyle.

### CROSS REFERENCE TO RELATED APPLICATIONS

This application is based upon and claims the benefit of priority from Japanese patent application No. 2018-105020, filed on May 31, 2018, the disclosure of which is incorporated herein in its entirety by reference.

### BACKGROUND

The present disclosure relates to a garment with biometric sensors attached thereto.

An apparatus that is produced by attaching biometric sensors to a vest-type garment in a scattered manner has been known (see, for example, Published Japanese Translation of PCT International Publication for Patent Application, No. 2014-505529). Electrodes of the biometric sensors are disposed in a flexible base-material layer that is formed so as to conform to the shape of the vest-type garment. Electrodes are connected to counterpart electrodes through curved strips provided in the base-material layer.

### SUMMARY

The present inventors have found the following problem. In the above-described related-art apparatus, there is no difficulty when the apparatus is used in a state in which a subject (i.e., a user) is at rest such as when the subject lies quietly to undergo an electrocardiograph test. However, in the related-art apparatus, when it is desired to acquire biological information in a state in which a subject is exercising and, in particular, when it is desired to test not only the body part (i.e., the trunk part) but also shoulders and upper limbs, there is a problem that the wiring material located in moving parts is repeatedly subject to stresses and hence it tends to be damaged.

An object of the present disclosure is to provide a durable garment with biometric sensors attached thereto, capable of making it possible to test a shoulder(s) and an upper limb(s) even when a subject is exercising.

A first exemplary aspect is a garment including a plurality of biometric sensors and adapted to cover a body part, a shoulder, and an upper limb of a subject, the plurality of biometric sensors being attached to the garment so that when the subject wears the garment each of them comes into contact with a respective one of a plurality of testing places, in which biometric sensors that are adjacent to each other across a shoulder joint are connected to each other by a wiring cable that passes above an acromial end, and biometric sensors that are adjacent to each other across an elbow joint are connected to each other by a wiring cable that passes an outer side of a lateral epicondyle. By connecting biometric sensors to each other by wiring cables and passing the wiring cables above the acromial end or the outer side of the lateral epicondyle according to the moving part of the subject as described above, the inventors have successfully suppressed the concentration of stresses and thereby improved the durability of the apparatus.

In the above-described garment, the plurality of biometric sensors disposed around the acromion end are preferably connected to each other by wiring cables that radially extend through a ring-shaped band disposed above the acromion end. It is possible to suppress an effect of expansion and contraction of a rotational motion of an arm around the shoulder joint by radially arranging the wiring cables around the acromial end as described above. That is, the wiring cables are hardly affected by circumferential expansion and contraction around the acromial end.

In the above-described garment, the biometric sensors may be detachable from respective attaching parts provided on cloth. Each of the attaching parts may have such a structure that a sensor unit included in the biometric sensor is attached from an inner side of the cloth and an amplifier unit included in the biometric sensor is attached from an outer side of the cloth with the cloth being sandwiched therebetween. By the above-described structure, the biometric sensors can be easily detached from the cloth, so that parts of the cloth that come into contact with skin of the subject can be washed. Further, the sensor units, which also come into contact with skin of the subject, can be easily disinfected.

Note that the wiring cables, which connect the biometric sensors to each other, are preferably connected to the amplifier units. Further, attaching forces are preferably adjusted so that when a user pulls the wiring cables, the amplifier units are detached from the counterpart sensor units. When the biometric sensors can be detached from the cloth just by grasping and pulling the wiring cables, which can be relatively easily grasped, efficiency of work performed by the user can be improved.

Further, the cloth of the garment is preferably elastic (or stretchable) so that the attached biometric sensors are brought into tight contact with the skin of the subject and the user can adjust positions of the biometric sensors by shifting them on the surface of the skin. When the cloth is elastic as described above, the subject can adjust the positions of the biometric sensors to appropriate positions after quickly wearing the apparatus. In addition, it is possible to bring the biometric sensors into tight contact with the skin in those places without requiring any special instrument. Therefore, the garment can be easily used and measurement can be immediately started.

Note that the cloth of the garment preferably includes a grasping part having a thickness larger than that of a surrounding part so that the user can adjust the positions of the biometric sensors by shifting them on the surfaces of the skin. By concentrating parts where the cloth is gripped into the grasping part having the large thickness, it is possible to reduce a possibility that the elastic cloth is damaged.

Further, the cloth of the garment may have a mark that should be positioned at a reference point in a skeletal structure of the subject. When there is the above-described mark, the subject can easily wear the garment by himself/herself even when there is no assistant having expert knowledge.

Further, the garment may include a ring-shaped band through which a wiring cable passes in a slidable manner in a passing point where the wiring cable passes. By using the above-described ring-shaped band, it is possible to stabilize a path of the wiring cable and to suppress the concentration of stresses even further.

Further, the garment is preferably a back-opening type garment in which an adjusting part adapted to adjust tightness in a chest is provided on a back side. When the garment is the back-opening type garment, the garment is worn in such a manner that both arms and the chest are simultaneously pressed against the garment. Therefore, the biometric sensors, which are arranged symmetrically in the horizontal direction, can be easily positioned and hence a wearing feeling is improved.

According to the present disclosure, it is possible to provide a durable garment with biometric sensors attached thereto, capable of making it possible to test a shoulder(s) and an upper limb(s) as well as a body part even when a subject is exercising.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a schematic diagram showing an overall configuration of a measurement system;

FIG. 2 shows a view of sensor wear worn by a subject as viewed from a chest side;

FIG. 3 shows a view of sensor wear worn by a subject as viewed from a back side;

FIG. 4 is a perspective view for explaining attachment of a biometric sensor to cloth; and

FIG. 5 is a perspective view for explaining a position adjustment of a biometric sensor.

### DESCRIPTION OF EMBODIMENTS

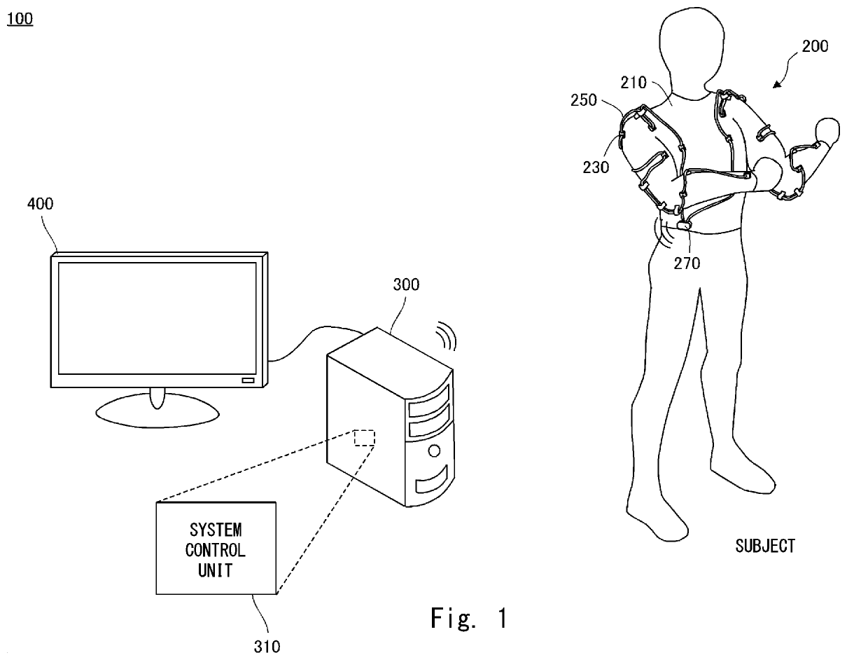

FIG. 1 is a schematic diagram showing an overall configuration of a measurement system 100 that measures myoelectric signals at a plurality of testing places on a subject. The measurement system 100 mainly includes sensor wear 200 (i.e., a sensor garment 200) worn by a subject, an analysis PC (Personal Computer) 300 that acquires myoelectric signals from the sensor wear 200 and analyzes a condition of the subject, and a monitor 400 that displays results of the analyses.

The sensor wear 200 is a garment to which biometric sensors 230 for acquiring myoelectric signals are attached in such a manner that when a subject wears the garment (i.e., the sensor wear 200), each of the biometric sensors 230 comes into contact with a respective one of a plurality of testing places on the subject. Further, its cloth 210 covers a body part (i.e., a trunk part), shoulders, and upper limbs of the subject. That is, the biometric sensors 230 are disposed on the cloth 210 so as to correspond to the testing places on the body part, the shoulders, and the upper limbs of the subject.

Biometric sensors 230 disposed on the right half of the body are connected to each other in a daisy-chain configuration by using wiring cables 250 wired from a control box 270. Further, Biometric sensors 230 disposed on the left half of the body are also connected to each other in a daisy-chain configuration by using wiring cables 250 wired from the control box 270. The routing (i.e., wiring) of the wiring cables 250 will be described later in detail.

An output signal from each of the biometric sensors 230 is collected in the control box 270 and transmitted to the analysis PC 300 through short-range radio communication. That is, the control box 270 includes a function of controlling each biometric sensor 230 and a function of communicating with the analysis PC 300. Further, the control box 270 has a function of supplying electric power to each biometric sensor 230. Therefore, the control box 270 includes a built-in secondary battery. Note that communication between the control box 270 and the analysis PC 300 is not limited to direct short-range radio communication. That is, the communication may be communication through a cloud server or communication through a cable.

The analysis PC 300 is, for example, a desktop PC, and functions as an analysis apparatus that receives output signals of the biometric sensors 230 from the control box 270 and carries out analyses related to the condition and/or the motion of the subject. The analysis PC 300 includes a system control unit 310 which may be a CPU (Central Processing Unit) and executes a control calculation program.

The monitor 400 is, for example, a liquid-crystal monitor and is connected to the analysis PC 300. Results of analyses by the analysis PC 300 and/or changes in the output signal of each biometric sensor 230 are displayed in the monitor 400 so that the subject and/or the assistant can visually recognize them.

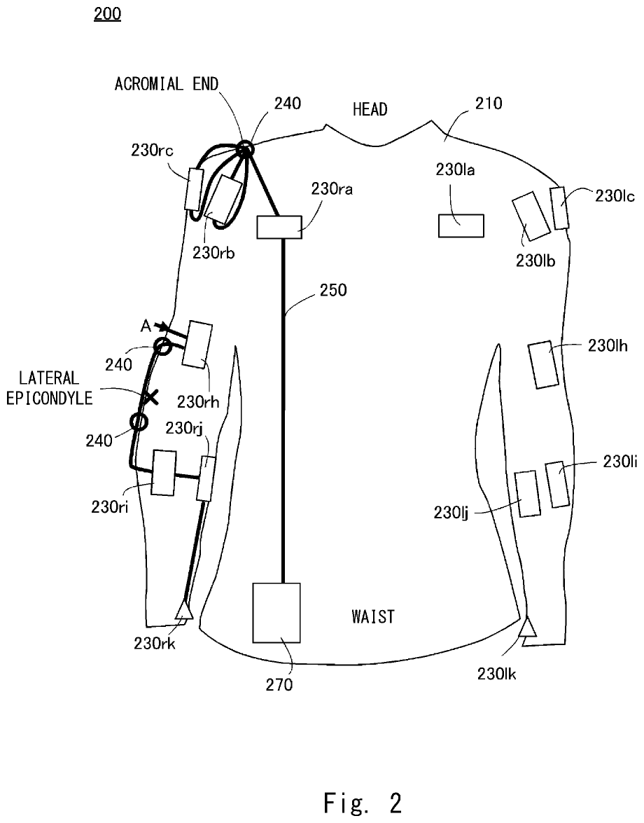

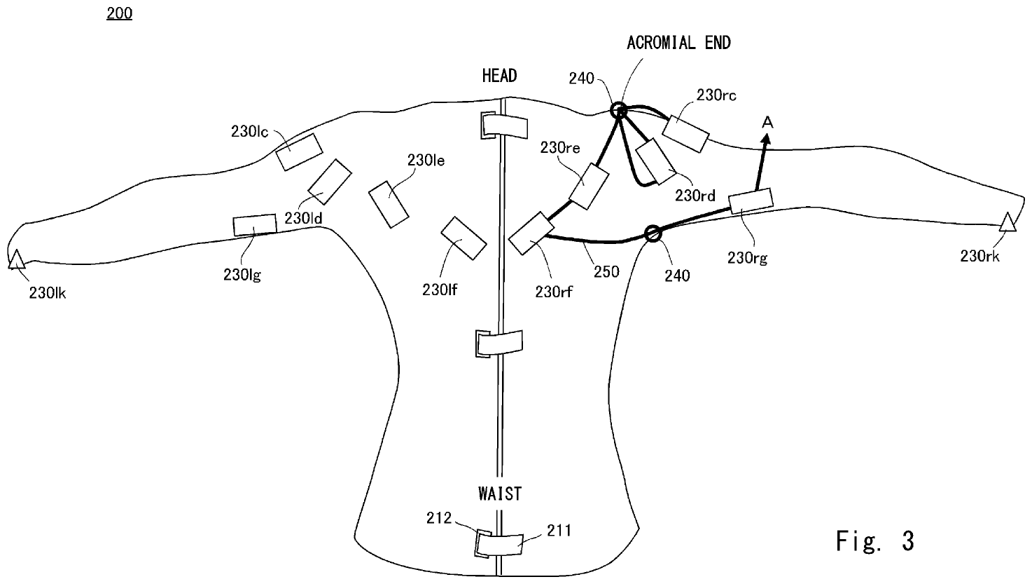

FIG. 2 is a simplified diagram of the sensor wear 200 worn by the subject as viewed from a chest side. Further, FIG. 3 is a simplified diagram of the sensor wear 200 worn by the subject as viewed from a back side. Although the wiring cables 250 and the ring-shaped bands 240 are shown only on the right half of the body, they are also disposed on the left half of the body substantially symmetric to those on the right half of the body.

As described above, each of the biometric sensors 230 is attached to the cloth 210. Firstly, a position of each biometric sensor 230 in a state where a subject wears the sensor wear 200 is described. When the subject wears the sensor wear 200, the position of each biometric sensor 230 corresponds to a respective one of testing places where it is desired to acquire myoelectric signals. Note that although examples of testing places are described hereinafter, the positions of the biometric sensors 230 can be changed as desired according to the purpose of the test.

Specifically, as the biometric sensors 230 disposed on the right half of the body, eleven biometric sensors are arranged. In particular, they are biometric sensors 230ra, 230rb, 230rc, 230rd, 230re, 230rf, 230rg, 230rh, 230ri, 230rj and 230rk, which are connected in this order from the control box 270. They are arranged on the right half of the body so that when the subject wears the sensor wear 200, the biometric sensor 230ra is positioned in a pectoralis major; the biometric sensor 230rb is positioned in a front part of a deltoid muscle; the biometric sensor 230rc is positioned in a middle part of the deltoid muscle; the biometric sensor 230rd is positioned in a rear part of the deltoid muscle; the biometric sensor 230re is positioned in an infraspinatus muscle; the biometric sensor 230rf is positioned in a trapezius muscle; the biometric sensor 230rg is positioned in a triceps brachii muscle; the biometric sensor 230rh is positioned in a biceps brachii muscle; the biometric sensor 230ri is positioned in a brachioradialis muscle; the biometric sensor 230rj is positioned in a flexor carpi radialis muscle; and the biometric sensor 230rk is positioned in an ulnar styloid process. Specifically, as the biometric sensors 230 disposed on the left half of the body, eleven biometric sensors are arranged. In particular, they are biometric sensors 2301a, 2301b, 2301c, 2301d, 2301e, 2301f, 2301g, 2301h, 2301i, 2301j and 2301k, which are connected in this order from the control box 270. They are arranged on the left half of the body so that when the subject wears the sensor wear 200, the biometric sensor 2301a is positioned in a pectoralis major; the biometric sensor 2301b is positioned in a front part of a deltoid muscle; the biometric sensor 2301c is positioned in a middle part of the deltoid muscle; the biometric sensor 2301d is positioned in a rear part of the deltoid muscle; the biometric sensor 2301e is positioned in an infraspinatus muscle; the biometric sensor 2301f is positioned in a trapezius muscle; the biometric sensor 2301g is positioned in a triceps brachii muscle; the biometric sensor 2301h is positioned in a biceps brachii muscle; the biometric sensor 2301i is positioned in a brachioradialis muscle; the biometric sensor 2301j is positioned in a flexor carpi radialis muscle; and the biometric sensor 2301k is positioned in an ulnar styloid process. Each of the biometric sensors 230 arranged on the left half of the body is disposed horizontally symmetric to a respective one of the biometric sensors 230 arranged on the right half of the body.

As described above, in the sensor wear 200, which covers the body part, the shoulders, and the upper limbs of the subject, places where considerable expansion and contraction and/or rotations could occur as the subject exercises are places corresponding to shoulder joints and places corresponding to elbow joints. The biometric sensors 230ra and 230rb have such a positional relation that they are adjacent to each other across the right shoulder joint. Each of the biometric sensors 230rb and 230rc, biometric sensors 230rc and 230rd, and biometric sensors 230rd and 230re also have such a positional relation that they are adjacent to each other across the right shoulder joint. Similarly, the biometric sensors 2301a and 2301b have such a positional relation that they are adjacent to each other across the left shoulder joint. Each of the biometric sensors 2301b and 2301c, biometric sensors 2301c and 2301d, and biometric sensors 2301d and 2301e also have such a positional relation that they are adjacent to each other across the left shoulder joint. Each of the wiring cables 250, which connect these biometric sensors with one another, is routed (i.e., wired) so as to pass above the right acromial end or above the left acromial end. By the above-described routing of the wiring cables 250, it is possible to prevent stresses from being concentrated on a certain wiring cable 250 or a certain part thereof, which would otherwise occur as the subject exercises.

Therefore, it is possible to make a possibility that the wiring cable 250 is broken extremely small.

More specifically, the biometric sensors 230ra, 230rb, 230rc, 230rd and 230re may be regarded as biometric sensors disposed around the right acromion end. These biometric sensors are connected to each other by the wiring cables 250 that radially extend through the ring-shaped bands 240 disposed above the right acromion end. Similarly, the biometric sensors 2301a, 2301b, 2301c, 2301d and 2301e may be regarded as biometric sensors disposed around the left acromion end. These biometric sensors are connected to each other by the wiring cables 250 that radially extend through the ring-shaped bands 240 disposed above the left acromion end. Each of the ring-shaped bands 240 is a loop-shaped band through which the wiring cable 250 passes in a slidable manner. For example, the ring-shaped band 240 is formed as follows. That is, one end of the ring-shaped band 240 is sewn to the cloth 210 and a hook-and-loop fastener (e.g., a Velcro) is disposed at the other end thereof, so that a loop having an appropriate diameter through which the wiring cable 250 passes can be formed. By radially arranging the wiring cables 250 as described above, it is possible to suppress an effect of expansion and contraction caused by a rotational motion of the arm around the acromial end. That is, since the wiring cables 250 are not wired along the circumferential direction around the acromial end, they are hardly affected by the expansion and contraction of the arm.

The biometric sensors 230rh and 230ri are adjacent to each other across the right elbow joint. The biometric sensors 2301h and 2301i are adjacent to each other across the left elbow joint. The wiring cables 250 connecting these sensors are routed so as to pass an outer side of the right lateral epicondyle and an outer side of the left lateral epicondyle, respectively. By the above-described routing of the wiring cables 250, it is possible to prevent stresses from being concentrated on a certain wiring cable 250 or a certain part thereof, which would otherwise occur as the subject exercises. Therefore, it is possible to make a possibility that the wiring cable 250 is broken extremely small.

Further, in order to make the wiring cables 250 pass the above-described predetermined places in a stable manner, the ring-shaped bands 240 are also disposed in several passing point. By disposing the ring-shaped bands 240 in several points, the wiring cables 250 pass the predetermined passing points and can be slid in these passing points. Therefore, it is possible to effectively release stresses, which would otherwise be exerted on the wiring cables 250, more effectively. The ring-shaped bands 240 are preferably disposed near the posterior walls of the axillae and on both sides of the outer sides of the lateral epicondyles as well as above the respective acromial ends.

Further, as shown in FIG. 3, the sensor wear 200 is back-opening type wear (i.e., a back-opening type garment) which is horizontally opened on the back side. Further, an adjusting part(s) for adjusting tightness in the chest is disposed so as to straddle the separating part of the back-opening type wear so that the cloth 210 is brought into tight contact with the body of the subject. Specifically, the adjusting part is formed by a combination of a fastening band 211 and a hook-and-loop fastener (e.g., a Velcro) 212. One end of the tightening band 211 is sewn to the right side of the back-opening type wear. The hook-and-loop fastener 212 is disposed on the left side of the back-opening type wear and is adapted so that the other end of the tightening band 211 is tightly attached thereto. It is possible to adjust the tightness in the chest by adjusting the attaching position of the other end of the tightening band 211.

When the sensor wear 200 has the back-opening structure as described above, a subject wears the sensor wear 200 so that both arms and the chest are simultaneously pressed against the sensor wear 200. Therefore, the biometric sensors, which are arranged symmetrically in the horizontal direction, can be easily positioned and hence a wearing feeling is improved. Further, since the tight contact between the biometric sensors 230 and skin of the subject can be improved by providing the adjusting part, accuracy of acquisitions of myoelectric signals can also be improved.

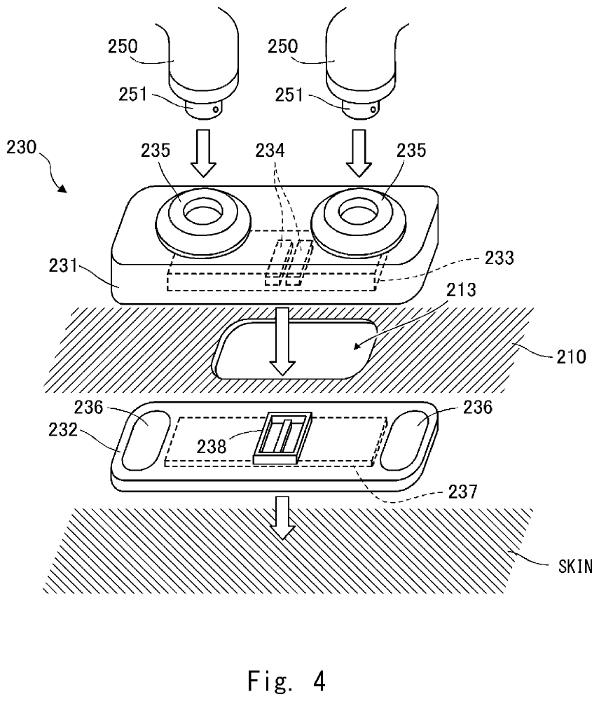

The biometric sensors 230 are detachable from the respective attaching parts provided on the cloth 210. FIG. 4 is a perspective view for explaining attachment of the biometric sensor 230 to the cloth 210. The biometric sensor 230 includes a sensor unit 232 and an amplifier unit 231. An attaching part 213, which is a through hole formed in the cloth 210, has such a structure that the sensor unit 232 is attached from the inner side of the cloth 210 (i.e., from the subject's skin side) and the amplifier unit 231 is attached from the outer side of the cloth 210 with a part of the cloth 210 located near the attaching part 213 being sandwiched therebetween.

On a surface of the amplifier unit 231 opposite to the surface thereof in contact with the cloth 210, two connection terminals 235 to which wiring cables 250 are connected are provided. The wiring cable 250 has a cable terminal 251 at its end and is fixed to the amplifier unit 231 by inserting this cable terminal 251 into the connection terminal 235. A wiring cable 250 having an appropriate length is selected (i.e., used) according to the distance between adjacent biometric sensors 230 or between the biometric sensor 230 and the control box 270. Note that in the case of the biometric sensors 2301k and 230rk, which are located at the ends of the daisy-chain configuration, a wiring cable 250 is connected to one of the connection terminals 235 but no wiring cable 250 is connected to the other connection terminal 235. Note that a terminator or the like may be connected to the other connection terminal 235 depending on the communication condition.

The connection terminal 235 is connected to an amplifier circuit 233 housed in the amplifier unit 231. The amplifier circuit 233 amplifies an acquired myoelectric signal, performs an AD conversion, and outputs a myoelectric signal processed according to a request from the control box 270. The amplifier circuit 233 is connected to an amplifier terminal 234 for acquiring a myoelectric signal from the sensor unit 232. The amplifier terminal 234 is disposed on a surface of the amplifier unit 231 opposed to the attachment unit 213. The amplifier terminal 234 is a female connector.

The sensor unit 232 contains a myoelectric sensor 237 that is exposed to the outside so that its electrode comes into contact with a skin of a subject. The myoelectric sensor 237 outputs a myoelectric signal, which is sensed through the skin of the subject, from a sensor terminal 238. The sensor terminal 238 is a male connector that is disposed on and protrudes from a surface of the sensor unit 232 opposed to the attachment unit 213. The sensor terminal 238 passes through the attaching part 213 and is received by the amplifier terminal 234. By the above-described terminal connections, the amplifier unit 231 and the sensor unit 232 are fixed to each other with the part of the cloth 210 located near the attaching part 213 being sandwiched therebetween. Further, the sensor unit 232 includes a magnet 236. As the magnet 236 sticks to a housing of the amplifier unit 231, the fixation between the amplifier unit 231 and the sensor unit 232 is reinforced.

As described above, each of the biometric sensors 230 is attached to a respective one of the attaching parts 213 disposed on the cloth 210. When a user, which may be a subject or an assistant, removes the biometric sensors 230 from the cloth 210, the user can detach the amplifier units 231 connected to the wiring cables 250 from the counterpart sensor units 232 by pulling the wiring cables 250. Since the amplifier units 231 are connected in series by the cable terminals 251, the user can simultaneously detach a lot of amplifier units 231. That is, a connecting force between the amplifier unit 231 and the sensor unit 232 is adjusted to such a degree that they are separated from each other as the user pulls one of them (or pulls them apart). Specifically, an engaging force between the amplifier terminal 234 and the sensor terminal 238 and/or the magnetic force of the magnet 236 are adjusted so that the above-described connecting force is obtained.

When the biometric sensors 230 and the wiring cables 250 can be easily pulled and detached from the cloth 210, the cloth 210, which comes into contact with skin of the subject, can be easily washed. Further, the sensor units 232, which also come into contact with skin of the subject, can be easily disinfected. Further, when the biometric sensors 230 can be separated from the cloth just by grasping and pulling the wiring cables 250, which can be relatively easily grasped, efficiency of work performed by the user can be improved.

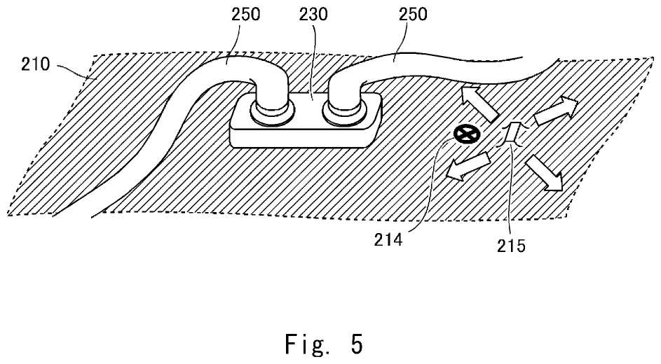

FIG. 5 is a perspective view for explaining a position adjustment of the biometric sensor 230. For the cloth 210, an elastic (or stretchable) material such as nylon is used. When the cloth 210 is elastic (or stretchable), it is expected that the cloth 210 presses the sensor unit 232 onto a skin of a subject. Therefore, it is possible to improve accuracy of acquisitions of myoelectric signals.

Since the cloth 210 is elastic, a user can adjust the position of the biometric sensor 230 by shifting it in a direction indicated by an arrow on the surface of the skin of the user. Note that in the cloth 210, a grasping part 215 having a thickness larger than that of a surrounding part is provided in the vicinity of the biometric sensor 230 whose position is to be adjusted. A user grasps (or pinches) this grasping part 215 and adjusts the position of the biometric sensor 230. A plurality of grasping parts 215 may be arranged in the vicinity of the biometric sensor 230. For example, two grasping parts 215 may be arranged on both sides of the biometric sensor 230. By concentrating parts where the cloth 210 is gripped into the grasping part 215 having the large thickness, it is possible to reduce a possibility that the elastic cloth 210 is damaged. Note that the grasping part 215 may be formed by folding a part of the cloth 210 or by attaching patch cloth on the cloth 210. Further, a knob made of plastic or metal, such as a button, may be attached on the cloth 210.

The cloth 210 may have a mark 214 that should be positioned at a reference point in a skeletal structure of a subject in the vicinity of the biometric sensor 230. In particular, in the case where the biometric sensors 230 are arranged in the vicinity of raised parts of the ulna and the scapula, when marks 214 are disposed at points on the cloth 210 that should be positioned at these raised parts, a user can easily position these biometric sensors 230.

The shape of the mark 214 is not limited to the one shown in the figure. That is, a letter, an icon, an illustration, etc. can also be used. For example, if an illustration of a skeleton or a muscle is drawn, it is possible to adjust its position so as to be positioned at the skeleton or the muscle of the subject. When the grasping part 215 and/or the mark 214 are disposed in the cloth 210, a subject can adjust the positions of the biometric sensors 230 to appropriate positions after wearing the sensor wear 200. In addition, it is possible to bring the biometric sensors 230 into tight contact with the skin in those places without requiring any special instrument. Therefore, the sensor wear 200 can be easily used and measurement can be immediately started.

Although the sensor wear 200 has been described as an example of a garment, the sensor wear 200 is not limited to those for measuring myoelectric signals. That is, by replacing the biometric sensors with other types of sensors, the sensor wear 200 can be configured to measure various biometric signals. Needless to say, the attaching parts 213 may be disposed at appropriate testing places according to the biometric signals to be measured. In the past, it has been a cumbersome task to individually place a number of biometric sensors in testing places and adjust their positions. In contrast, by using sensor wear like the one described in this embodiment, it is possible to dispose a number of biometric sensors at predetermined positions by just having a subject wear the sensor wear.

Further, although the above-described sensor wear 200 is configured as a back-opening type garment, it may be formed as a front-opening type garment or a poncho type garment. An appropriate configuration may be selected (i.e., used) while taking, for example, the arrangement and positions of biometric sensors 230 into consideration. Further, the above-described biometric sensor 230 has been described under the assumption that the biometric sensor 230 and the wiring cable 250 are detachable. However, the present disclosure is not limited such configurations By configuring the biometric sensor 230 and the wiring cable 250 so that they are detachable, the connection configuration of the biometric sensor 230 and the wiring cable 250 can be changed as appropriate according to the testing place to be measured. On the other hand, by configuring the biometric sensor 230 (or the amplifier unit 231 included therein) and the wiring cable 250 so that they are fixed to each other, it would be more convenient in the case where predetermined measurement is repeated.

Further, in the sensor wear 200, the daisy-chain configuration in which the biometric sensors 230 are connected in series is used. This is because it is possible to reduce the thickness of the wiring cables 250 as compared to the case where they are connected in parallel, and hence it is less likely to interfere with the exercise performed by the subject. However, the wiring configuration is not limited to the daisy-chain configuration as long as the thickness of the wiring cables can be reduced. However, even in this case, the biometric sensors that are adjacent to each other across the shoulder joint are preferably connected to each other by a wiring cable that passes above the acromial end. Further, the biometric sensors that are adjacent to each other across the elbow joint are preferably connected to each other by a wiring cable that passes the outer side of the lateral epicondyle.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A garment comprising a plurality of biometric sensors and adapted to cover a body part, a shoulder, and an upper limb of a subject, the plurality of biometric sensors being attached to the garment so that when the subject wears the garment each of them comes into contact with a respective one of a plurality of testing places, wherein
biometric sensors that are adjacent to each other across a shoulder joint are connected to each other by a wiring cable that passes above an acromial end, and biometric sensors that are adjacent to each other across an elbow joint are connected to each other by a wiring cable that passes an outer side of a lateral epicondyle.

2. The garment according to claim 1, wherein the plurality of biometric sensors disposed around the acromion end are connected to each other by wiring cables that radially extend through a ring-shaped band disposed above the acromion end.

3. The garment according to claim 1, wherein
the biometric sensors are detachable from respective attaching parts provided on cloth, and
each of the attaching parts has such a structure that a sensor unit included in the biometric sensor is attached from an inner side of the cloth and an amplifier unit included in the biometric sensor is attached from an outer side of the cloth with the cloth being sandwiched therebetween.

4. The garment according to claim 3, wherein the wiring cables, which connect the biometric sensors to each other, are connected to the amplifier units, and attaching forces are adjusted so that when a user pulls the wiring cables, the amplifier units are detached from the counterpart sensor units.

5. The garment according to claim 1, wherein the cloth of the garment is elastic so that the attached biometric sensors are brought into tight contact with the skin of the subject and the user can adjust positions of the biometric sensors by shifting them on the surface of the skin.

6. The garment according to claim 5, wherein the cloth of the garment comprises a grasping part having a thickness larger than that of a surrounding part so that the user can adjust the positions of the biometric sensors by shifting them on the surface of the skin.

7. The garment according to claim 1, wherein the cloth of the garment has a mark that should be positioned at a reference point in a skeletal structure of the subject.

8. The garment according to claim 1, further comprising a ring-shaped band through which a wiring cable passes in a slidable manner in a passing point where the wiring cable passes.

9. The garment according to claim 1, wherein the garment is a back-opening type garment in which an adjusting part adapted to adjust tightness in a chest is provided on a back side.
